(19) United States
(12) Patent Application Publication
(10) Pub. No.: US 2018/0021575 A1
(43) Pub. Date: Jan. 25, 2018

(54) UNIVERSAL METHOD FOR PREVENTING FALLING ASLEEP AND IMPROVING HUMAN PERFORMANCE

(71) Applicant: Ivan Rybachenko, Kharkiv (UA)

(72) Inventor: Ivan Rybachenko, Kharkiv (UA)

(21) Appl. No.: 15/448,624

(22) Filed: Mar. 3, 2017

Related U.S. Application Data
(60) Provisional application No. 62/366,258, filed on Jul. 25, 2016.

(51) Int. Cl. A61N 1/36; A61N 1/04

(52) U.S. Cl. CPC A61N 1/36014; A61N 1/0472

(57) ABSTRACT

A method for preventing falling asleep and improving human performance, comprising: impacting on human skin with regular electrical impulses and receiving such impact, wherein the regular electrical impulses are fed at short intervals of equal or random length that is adjusted to change the intensity of exposure. Also disclosed a compact device such as a wrist watch or bracelet for providing preventing falling asleep and improving human performance, comprising: a voltage source, a control system and electrodes adjacent to the skin.

### TECHNICAL FIELD

The present invention relates to the field of health and rescue of human life.

### BACKGROUND

Unwanted falling asleep and losing control of consciousness is a global problem that causes damage and creates risks for every active person. In the context of information overload, such people rest less and less and often fall asleep at work, which may lead to disasters. Moreover, it seems to be a sign of weakness and serves as a cause for ridicule (especially for public personalities) in everyday life.

The need for a universal, effective and reliable device to prevent this issue is long overdue, but the task has never been accomplished. Although a variety of anti-sleeping devices and patents is known in this field, they have not coped with the task and have not been spread because of a fundamental flaw in the approach to solving the problem. They used a scheme that detects the event of falling asleep through sensors of various vital functions and the subsequent reaction (alarm signal). These devices would come out bulky, unreliable, requiring complicated calibration, with frequent failures and, as a result, have not achieved recognition and extensive use.

The provided method makes it possible to eliminate all these drawbacks and create an effective, universal, reliable device to solve this problem.

### DESCRIPTION OF THE PRIOR ART

Devices useful as somnolence detection and drowsiness warning system for preventing accidents due to a vehicle operators falling asleep are known in the art. U.S. Pat. No. 6,822,573 to Bashir et al describes a non-intrusive system used to determine if the driver of a vehicle is drowsy and at risk of falling asleep at the wheel due to drowsiness. The system consists of two different drowsiness detection systems and a control unit. This redundancy reduces the risk of a false drowsiness assessment. The first subsystem consists of an array of sensors, mounted in the vehicle headliner and seat, which detects head movements that are indicative characteristics of a drowsy driver. The second subsystem consists of heart rate monitoring sensors placed in the steering wheel. The control unit is used to analyze the sensory data and determine the driver's drowsiness state and therefore corresponding risk of falling asleep while driving. Through sensory fusion, intelligent software algorithms, and the data provided by the sensors, the system monitors driver characteristics that may indicate a drowsy driver. If the driver is found to be drowsy, a signal is outputted which may be used to activate a response system. This system is not limited to automobiles; this system may be used in any type of vehicle, including aircrafts, trains and boats.

U.S. Pat. No. 6,337,629 to Bader discloses and claims a method and a system for monitoring an individual's autonomic nervous system in order to detect when that individual's level of wakefulness decreases. Changes detected in the stimuli of the autonomic nervous system are reflected by changes in the behaviors of certain physiological parameters. Sensors attached to the body of the driver measure respiration, cardiac activity and body movements and any reduction of the cardiac or pulmonary parameters. The sensors can be of any type and are preferably a conventional sensor such as PVP type sensors. The sensors are attached to a data collecting computer unit and further to either a light emitting device, a siren or a vibrating device which is activated when lower heart rates are measured.

U.S. Pat. No. 6,575,902 to Burton et al discloses and claims a device for monitoring the “awareness” or vigilance of an individual operating a vehicle or the like. The apparatus includes a number of sensor means such as electroencephalograms (EEG), electro-oculograms (EOG) or electro-myelograms (EMG) which, when monitoring live physiological parameters below a predetermined threshold level, will turn off the vehicle and/or trigger an additional vibratory or sound device to wake up the otherwise drowsy individual.

U.S. Pat. No. 6,147,612 to Ruan et al teaches a duel function optic sleep prevention device to prevent drivers of vehicles from falling asleep behind the wheel comprising a micro-optic eyelid monitor, a micro-optic tilting monitor, a signal combination circuits and an alarming unit. The eyelid monitor is placed in the vicinity of an eye by an appropriate mounting device when in use, using an emitter to transmit modulated appropriate light signals towards the eye, and using optic sensors to sense the influenced light by the eyelid, to detect the movement of eyelid. The tilting monitor comprises an optic tilt switch set on the side head of a user, using a micro-optic monitor to detect a small ball's rolling in a closed tube system inside the switch when tilting. Both the monitors are connected to signal combination circuits, which have an input signal filtering device, a switch part and a counter. The filtering device sets an appropriate time delay range to pick up dangerous long symptoms, based on signal length and the difference between a monitor signal's appearing time and another monitor's. The counter circuits record the time of those signals' occurrence. When finding such a symptom, the combination circuits will stimulate a pre-alarming immediately to detect the sleepy state of a user. A fully alarming will follow if the user has no response after a limited waiting time, or if the time of repeating pre-alarming reaches a criteria value set in the counter. U.S. Pat. No. 6,154,123 to Kleinberg teaches a driver alertness monitoring system. A driver alertness monitoring system for motor vehicles which issues visual or audible signals to the driver, and which monitors visual, audible or mechanical responses received from the driver to determine driver alertness. An electronic control unit, preferably incorporated in the circuitry of existent vehicle safety systems, controls the activation of these visual or audible signals and determines appropriate actions based on the response of the driver. The system includes auxiliary sensors, which can be used to control and modify the function of the system to reduce unnecessary activation.

U.S. Pat. No. 6,734,799 to Munch describes an apparatus that monitors the health and fitness of a motor vehicle driver comprising a number of sensor means, communicating means, transmitting means and responder means operatively connected in tandem for reporting a health factor or condition of an individual outside of the norm and means for responding thereto. The apparatus may also be connected to the drive mechanism of the vehicle so as to be able to shut it off if exigent circumstances require.

U.S. Pat. No. 5,807,267 to Bryars et al discloses a pulse rate monitor for sensing a pulse wave produced at an arterial pulse source of the wearer. Piezo pressure sensors or optical sensors outside the surface of the skin produce an electrical signal upon detecting the presence of a pulse in the artery. When using the piezo sensors, a second (background) sensor is employed. Signals from this background sensor are digitally subtracted from the primary pulse sensor thus significantly reducing the effects of body motion signals. Optical sensor configurations typically use only a single sensor. Signals from the sensors are amplified and passed through appropriate filters to reduce the bandwidth of the input circuitry to pass only the signals of interest. The filtered signals are converted to digital signals in a microprocessor. These signals are then digitally processed to produce an output on the display in pulses per minute. The unit may be worn in the same manner as a wrist watch and powered from a small battery. Some embodiments of this device may have all the time keeping capability of a digital wrist watch.

Finally, U.S. Pat. No. 5,846,206 to Boder discloses and claims a device for monitoring and estimating a person's wakefulness comprising a stationary pressure sensor that contacts part of that person's body and generates a signal corresponding to that person's body movements relative to the sensor. A detector/filter circuit connected to the sensor separates signals corresponding to true body movements, heartbeat-related body movements, and respiration-related body movements. Calculation and comparison circuits are adapted to receive these signals and compare them with previously received signals to determine possible changes. A correlator correlates the output signals from the calculation and comparison circuits to produce a resulting change signal, which is compared with a preset wakefulness threshold in a threshold detector circuit. An alarm device is triggered, when the preset threshold is achieved as an indication of a reduced degree of wakefulness.

All these and other patents suggest devices to counter sleepiness, which rely on sensors such as heart rate or eye blinking frequency sensors. In practice, these devices do not meet the expectations of users since their equipment is often mistaken, captures only the later symptoms of sleepiness and has no effect on their cause.

### BRIEF SUMMARY OF THE INVENTION

In accordance with the present invention, the problems identified in the prior art are solved by providing a method for preventing falling asleep and improving human performance, comprising: impacting on human skin with regular electrical impulses and receiving such impact, wherein the regular electrical impulses are fed at short intervals of equal or random length that is adjusted to change the intensity of exposure. In accordance with the present invention, the problems identified in the prior art are also solved by developing a compact device such as a wrist watch or bracelet for providing preventing falling asleep and improving human performance, comprising: a voltage source, a control system and electrodes adjacent to the skin.

This summary is presented for familiarization with the inventive concept in a simplified form, which is further disclosed under Detailed Description of the Invention. This Summary of the Invention is not intended to define dominant or essential features of the subjects of the present invention as well as to limit the scope of the invention.

### DETAILED DESCRIPTION AND BEST EMBODIMENT OF THE INVENTION

As discussed above, to solve the existing problem evident in the prior art, there is provided a method for preventing falling asleep and improving human performance, comprising: impacting on human skin with regular electrical impulses and receiving such impact, wherein the regular electrical impulses are fed at short intervals of equal or random length that is adjusted to change the intensity of exposure. The regular electrical impulses provide a tactile sensation of discomfort or slight pain. Receiving such an impact triggers mobilization of the nervous system and a sharp reduction of sleepiness symptoms. Our approach, on the contrary, affects firstly the cause of sleepiness and does not depend on the prominence of its symptoms. This makes it possible to improve the reliability by several times, exclude the factor of individual characteristics, remove bulky sensors and redundant nodes as well as minimize the size of the device and its final cost.

According to the invention there is also provided a compact device such as a wrist watch or bracelet for providing preventing falling asleep and improving human performance, comprising: a voltage source, a control system and electrodes adjacent to the skin.

In one of the preferred embodiments of the present invention, the device further comprises a button, said button being configured to activate the devices manually by pressing it.

In another embodiment of the present invention, the device further comprises: a sensor, said sensor being configured to activate automatically through a sensor signal.

In another preferred embodiment of the present invention, the device is configured to generate regular electrical impulses and to adjust current intensity, voltage and impulse length so as to achieve a more pronounced effect. Receiving such an impact triggers mobilization of the nervous system and a sharp reduction of sleepiness symptoms, while unwanted falling asleep is almost impossible. (Rybachenko I. and PhD Luzenko E. study this phenomenon in the “Research of a method of cyclic electrical influence for prevention paroxysms of a sleepy condition in conditions of monotonous activity”, article in Psychophysiology News, 2014, St. Petersburg. (https://drive.google.com/file/d/0B4fqUCEDRbz0NUkySEV2ZzVhZEU/view)) According to the invention the electrical impulses of a certain form can be transmitted on the user's skin in multiple speed modes and with intensity of several modes. The intensity of the impulse can be selected by the user from multiple levels, the strength of which is determined by experimentation in a safe current range with a notional load and tested.

The device triggers annoying, and at the highest intensity, for example some pain, due to which the user selects himself. When the body experiences some pain, it is waiting for the next discharge, straining, and there is the noradrenaline rush with such consequences: general mobilization of consciousness, improved supply the brain with oxygen, the disappearance of drowsiness. Practical tests using the device showed a reduction in symptoms of sleepiness about 70%/o.

1. A method for preventing falling asleep and improving human performance, comprising:
impacting on human skin with regular electrical impulses and receiving such impact, wherein the regular electrical impulses are fed at short intervals of equal or random length that is adjusted to change the intensity of exposure.

2. A compact device for providing preventing falling asleep and improving human performance, comprising:
a voltage source, a control system and electrodes adjacent to the skin.

3. The compact device according to claim 2, further comprising a button, said button being configured to activate the devices manually by pressing it.

4. The compact device according to claim 2, further comprising a sensor, said sensor being configured to activate automatically through a sensor signal.

5. The compact device according to claim 2, wherein the device is configured to generate regular electrical impulses and to adjust current intensity, voltage and impulse length to achieve a more pronounced effect.
